(19) United States
(12) Patent Application Publication
(10) Pub. No.: US 2007/0027136 A1
(43) Pub. Date: Feb. 1, 2007

(54) Formulations of anthraquinone derivatives

(76) Inventors: Gavin William Halbert, Glasgow (GB); Steven John Ford, Glasgow (GB); Moira Alexandra Elliott, Lanarkshire (GB)

Correspondence Address: NIXON & VANDERHYE, PC, 901 NORTH GLEBE ROAD, 11TH FLOOR, ARLINGTON, VA, 22203, US

(21) Appl. No.: 10/570,500

(22) PCT Filed: Sep. 16, 2004
(86) PCT No.: PCT/GB04/03954

§ 371 Date: Mar. 3, 2006

(30) Foreign Application Priority Data
Sep. 17, 2003 (GB) 0321787.4
Dec. 23, 2003 (GB) 0329875.9

(51) Int. Cl. A61K 31/55; A61K 31/445; A61K 31/40; A61K 31/135

(52) U.S. Cl. 514/212.01; 514/656; 514/319; 514/408

(57) ABSTRACT

A stable, sterile aqueous solution of a compound of formula (I): in which A is a C alkylene group with a chain length between NH and N(O)R′R″ of at least 2 carbon atoms and R′ and R″ are each separately selected from C₁₋₄ alkyl groups and C₂₋₄ hydroxyalkyl and C₂₋₄ dihydroxyalkyl groups, or R′ and R″ together are a C₂₋₆ alkylene group, is formulated in a unit dosage form in a sealed container, said solution having a concentration of the compound of formula (I) up to 150 mg/ml and a pH in the range of 5 to 9. The solution may be prepared without a freeze drying step.

The invention relates to novel formulations of anthraquinone derivarives such as AQ4N, a bis-bioreductive agent with value in the treatment of cancer.

WO-A-91/05824 (National Research Development Corporation) discloses a compound of formula: 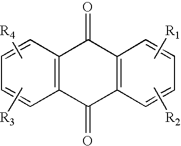
in which R₁, R₂, R₃ and R₄ are each separately selected from hydrogen, X, NH-A-NHR and NH-A-N(O)R′R″ wherein X is hydroxy, halogeno, amino, C₁₋₄ alkoxy or C₂₋₄ alkanoyloxy, A is a C alkylene group with a chain length between NH and NHR or N(O)R′R″ of at least 2 carbon atoms and R, R′ and R″ are each separately selected from C₁₋₄ alkyl groups and C₂₋₄ hydroxyalkyl and C₂₋₄ dihydroxyalkyl groups in which the carbon atom attached to the nitrogen atom does not carry a hydroxy group and no carbon atom is substituted by two hydroxy groups, or R′ and R″ together are a C₂₋₆ alkylene group which with the nitrogen atom to which R′ and R″ are attached forms a heterocyclic group having 3 to 7 atoms in the ring, the compound optionally being in the form bf a physiologically acceptable salt.

A preferred compound within this general formula is the bis-N-oxide AQ4N, (banoxantrone) which may be synthesised by oxidation of AQ4:

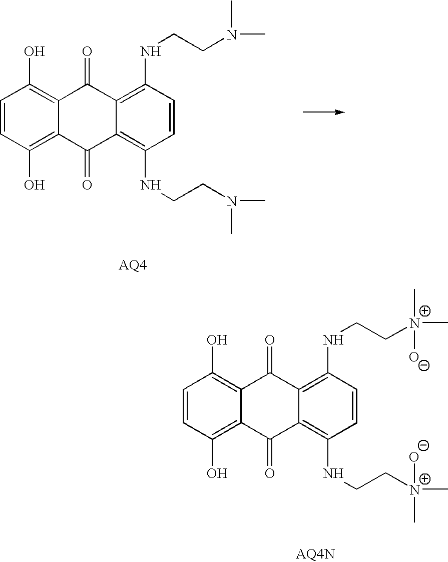

AQ4N is in fact a prodrug and the reverse reaction occurs in vivo, reductive metabolism in hypoxic cancer cells giving the active agent, AQ4, in its protonated form. The prodrug is relatively non-toxic when compared with the active agent, AQ4, making it particularly attractive for administration as a pharmaceutical. However, it does not readily give a crystalline form. Up to now, the thinking has been that it is desirable to prepare and formulate AQ4N for administration in the form of a salt or freeze dried material.

AQ4N has been reported in the form of a dihydrochloride salt AQ4N.2HCl. See for example J. Chem. Soc., Perkin Trans. I, 1999, 2755-2758 (Lee et al.) and WO-A-00/05194 (BTG International Limited). However, investigations of AQ4N.2HCl raw material have demonstrated significant quantities of an impurity 1-amino-4-{[2-(dimethylamino)ethyl]amino}-5,8-dihydroxyanthraquinone, denoted AQMN, which has been characterised by LCMS and also confirmed by synthesis of a genuine sample of AQMN material:

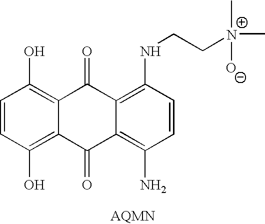

This impurity can be formed by degradation of AQ4N, and more significantly shows an undesirable level of cytotoxicity, generally being higher than that of AQ4N itself. This level of cytotoxicity is to be avoided in a compound which is intended to be administered in the form of a relatively non-toxic prodrug.

Although the AQMN degradation is the predominant pathway, a further degradation product of AQ4N under acidic and neutral aqueous solution conditions is a further undesirable product, namely the mono-N-oxide, AQ4M:

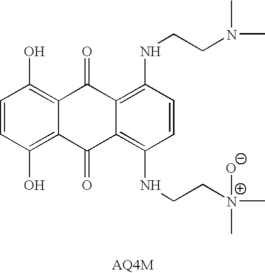

Co-pending patent application WO-A-03/078387 (BTG International Limited) discloses further salts of AQ4N, with a physiologically acceptable acid having a pKa in the range of −3.0 to 9.0. The patent application also discloses the formulation of the compound so that upon dissolution in aqueous solution the pH of the solution is in the range of 5 to 9.

After manufacture of a pharmaceutical product, the point at which it expires (and can no longer be used in patients) is defined by the decrease in the content of the intended drug and/or the increase in associated impurities. A reduction in the degradation rate of the parent compound increases the time it takes for that compound to reach the point at which it expires (the expiry date). Where required, drug compounds are stabilised by producing the final product as a freeze dried formulation.

However with AQ4N the present inventors have found that the stability and manufactured quality of AQ4N as a freeze dried formulation is inversely related to the water content of the freeze dried cake: that is, the less water, the poorer the quality and stability of the final product. Moreover, it is more stable as a solution (at the appropriate pH) than as the equivalent freeze dried formulation. Thus it is better to produce AQ4N without freeze drying.

Thus according to the present invention there is provided a stable, sterile aqueous solution of a compound of formula (I): 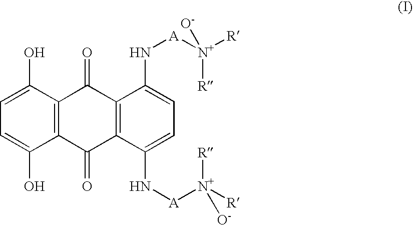
in which A is a C alkylene group with a chain length between NH and N(O)R′R″ of at least 2 carbon atoms and R′ and R″ are each separately selected from C₁₋₄ alkyl groups and C₂₋₄ hydroxyalkyl and C₂₋₄ dihydroxyalkyl groups in which the carbon atom attached to the nitrogen atom does not carry a hydroxy group and no carbon atom is substituted by two hydroxy groups, or R′ and R″ together are a C₂₋₄ alkylene group which with the nitrogen atom to which R′ and R″ are attached forms a heterocyclic group having 3 to 7 atoms in the ring,

in a unit dosage form in a sealed container, said solution having a concentration of the compound of formula (I) up to 150 mg/ml and a pH in the range of 5 to 9.

Preferably the pH of the solution is in the range of 5.0 and 8.4, more preferably 6.0 to 8.0, with a pH between 7.0 and 8.0 being optimal. When the compound of formula (I) is AQ4N, a pH 7.4 has been shown to be the most stable. This contrasts with the freeze dried AQ4N formulation, where the most stable was observed to be pH 6.

Preferably the compound of formula (I) is present at a concentration of between 0.1 and 100 mg/ml.

The compound of formula (I) may be previously isolated in the form of a physiologically acceptable salt which will be an acid addition salt with an organic or inorganic acid. Preferably the physiologically acceptable acid has a pKₐ in the range of −3.0 (minus 3.0) to 9.0, and more preferably in the range of 2.0 to 9.0. More preferably the physiologically acceptable acid has a pKₐ in the range of 2.0 to 6.0.

Preferably the physiologically acceptable acid is selected from the group consisting of tartaric acid, malonic acid, dichloroacetic acid, citric acid, maleic acid, benzenesulfonic acid, pimelic acid and acetic acid.

More preferably the physiologically acceptable acid has a pKₐ in the range of 3.0 to 6.0. The physiologically acceptable acid may especially be an organic acid, particularly an organic mono-, di- or tri-acid, and especially one selected from the group consisting of tartaric acid, citric acid, pimelic acid and acetic acid.

The group A in formula (I) may be branched but is conveniently a straight chain alkylene group, i.e. tetramethylene, especially trimethylene, or particularly ethylene.

R′ and R″ may also have a branched carbon chain but are conveniently straight chain whether they are alkyl groups or hydroxy-substituted alkyl groups. When R′ or R″ is a monohydroxyalkyl group this is conveniently substituted terminally and when R′ or R″ is a dihydroxyalkyl group this is conveniently substituted terminally by one of the hydroxy groups. When R′ and R″ are alkyl the preference is for a group of three or especially two or one carbon atoms and when R′ and R″ are hydroxy-substituted alkyl the preference is for the alkyl group to be of three carbon atoms or, in the case of a monohydroxyalkyl group, alternatively of two carbon atoms. Examples of preferred individual groups R′ and R″ are CH₃, CH₂CH₃, CH₂CH₂CH₃, CH₂CH₂OH, CH₂CH₂CH₂OH, CH(CH₃)CH₂OH and CH₂CHOHCH₂OH.

R′ and R″ will more usually be identical.

Alternatively, as indicated, R′ and R″ together with the nitrogen atom to which they are attached may represent a heterocyclic group —N(CH₂)ₙ where n is 2 to 6, i.e. aziridin-1-yl, azetidin-1-yl, pyrrolidin-1-yl, piperidin-1-yl and perhydroazepin-1-yl, the smaller groups such as azetidin-1-yl and especially aziridin-1-yl being of most interest.

Specific groups NH-A-N(O)R′R″ of particular interest are NH—(CH₂)₂—N(O)(CH₃)C₂H₅, NH—(CH₂)₂—N(O)(C₂H₅)₂, NH—(CH₂)₂—N(O)(CH₂CH₂OH)₂, NH—(CH₂)₂—N(O)(CH₂CH₂CH₂OH)₂, NH—(CH₂)₂—N(O)(CH(CH₃)CH₂OH)₂, NH—(CH₂)₂—N(O)(CH₂CHOHCH₂OH)₂ and especially NH—(CH₂)₂—N(O)(CH₃)₂.

The physiologically acceptable salt when simply dissolved in aqueous solution will normally give a solution having a pH lower than the desired range. For example, the acetate salt of AQ4N in a 1.4 millimolar solution aqueous solution has a pH of 3.8. Thus preferably the compound of formula (I) is formulated in a mixture containing additional components so the pH of the solution is buffered to be in the range of 5 to 9.

A buffer is a solvated mixture of salt and acid, which oppose changes in pH when small amounts of acid and bases are added to the solution. Suitable buffers include sodium acetate buffer and sodium orthophosphate buffer.

The salt with a physiologically acceptable acid may be prepared by any conventional means, for example by reaction of the organic base (1) with the appropriate inorganic or organic acid, usually by simple admixture in solution. The acid addition salts are generally crystalline solids which are relatively soluble in water, methanol, ethanol and similar solvents. One salt form may also be converted into another by chromatography using a column which has been pre-treated with the desired physiologically acceptable acid.

The compound of formula (I) may be formulated with a physiologically acceptable diluent or carrier for use as pharmaceuticals for both veterinary and particularly human use by a variety of methods. For instance, it may be applied as a composition incorporating a liquid diluent or carrier, for example an aqueous solution, suspension or emulsion, which may often be employed in injectable form for parenteral administration and therefore may conveniently be sterile and pyrogen free. Oral administration may also be used.

Other types of administration than by injection or through the oral route which are of use in both human and veterinary contexts include the use of suppositories or pessaries. Another form of pharmaceutical composition is one for buccal or nasal administration or alternatively drops for administration into the eye which may conveniently contain a sterile liquid diluent or carrier. Other formulations for topical administration include lotions, ointments, creams, gels and sprays.

Compositions may be formulated in unit dosage form, i.e. in the form of discrete-portions containing a unit dose, or a multiple or sub-unit of a unit dose.

Whilst the dosage of the compound used will vary according to the activity of the particular compound and the condition being treated, it may be stated by way of guidance that a dosage selected in the range from 25-500 mg/m² per day, particularly in the range from 50-300 mg/m² per day, will often be suitable although higher doses than this, for example in the range from 25-750 mg/m² per day, or even doses up to 1200 mg/m², may be considered in view of the lower level of toxic side effects obtained with the compounds (I). This dosage regime may be continued for however many days is appropriate to the patient in question, the daily dosages being divided into several separate administrations if desired. Thus, for example, in the case of conditions such as advanced breast cancer, non-Hodgkin's lymphoma and hepatoma, treatment for one day followed by a repeated dose after an interval, such as 21 days, may be appropriate whilst for the treatment of acute non-lymphocytic leukaemia, treatment over 5 consecutive days may be more suitable. Alternatively, single administrations spaced by several days, for example one dose every two or three weeks, may be used.

The compounds (I) are of particular value for the treatment of cancer in warm blooded animals including humans. The compounds are of interest in relation to the treatment of solid tumours, such as various forms of sarcoma and carcinoma, and also for disseminated tumours such as leukaemias. Areas of particular interest are the treatment of breast cancer, lung cancer, prostate cancer, pancreatic cancer, and oesophageal cancer, and the treatment of non-Hodgkin's lymphoma and acute non-lymphocytic leukaemia. In the treatment of cancer, parenteral and sometimes topical administration is often of particular interest. Moreover, it may be advantageous to use the compounds (I) in a combined treatment, given separately or together in the same composition, with other anti-cancer agents, such as mitotic inhibitors, for example vinblastine; alkylating agents, for example cisplatin, carboplatin and cyclophosphamide; other antimetabolites, for example 5-fluorouracil, cytosine arabinoside and hydroxyurea; intercalating antibiotics, for example adriamycin and bleomycin; enzymes, for example asparaginase; topoisomerase inhibitors, for example etoposide and biological response modifiers, for example interferon. The compounds (I) may also be used in combined treatment with radiation therapy of the tumour.

The present invention thus includes a method for aiding regression and palliation of cancer which comprises administering to a patient a therapeutically effective amount of a compound (I) as defined hereinbefore.

In addition to their anti-cancer use the compounds (I) are of interest for various other pharmaceutical applications in view of their activity as chelating agents.

The present invention also includes a process for the preparation of a solution in a unit dosage form in a sealed container as defined above, comprising introducing a stable, sterile aqueous solution of the compound of formula (I) into a container and sealing the container, in which the solution is prepared without a freeze drying step.

The invention is illustrated by the following Examples in which

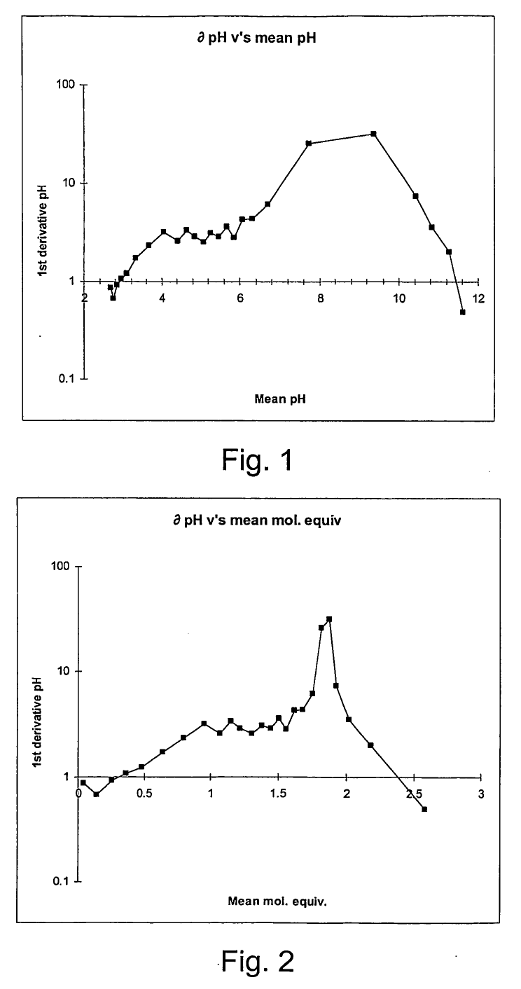

FIG. 1 shows the first derivative of pH versus pH in a solution of AQ4N dihydrochloride;

FIG. 2 shows the first derivative of pH versus NaOH molar equivalence under the same conditions;

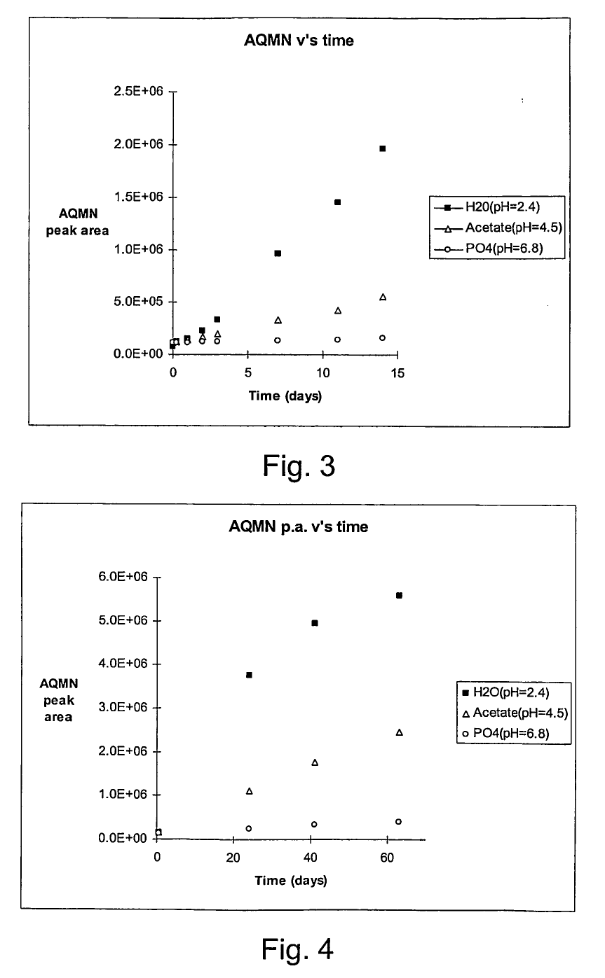

FIG. 3 shows the increase in AQMN over incubation time in 5 mg/ml solutions incubated at 40° C. for 14 days;

FIG. 4 shows the increase in AQMN over incubation time in 5 mg/ml solutions incubated at 40° C. for 63 days;

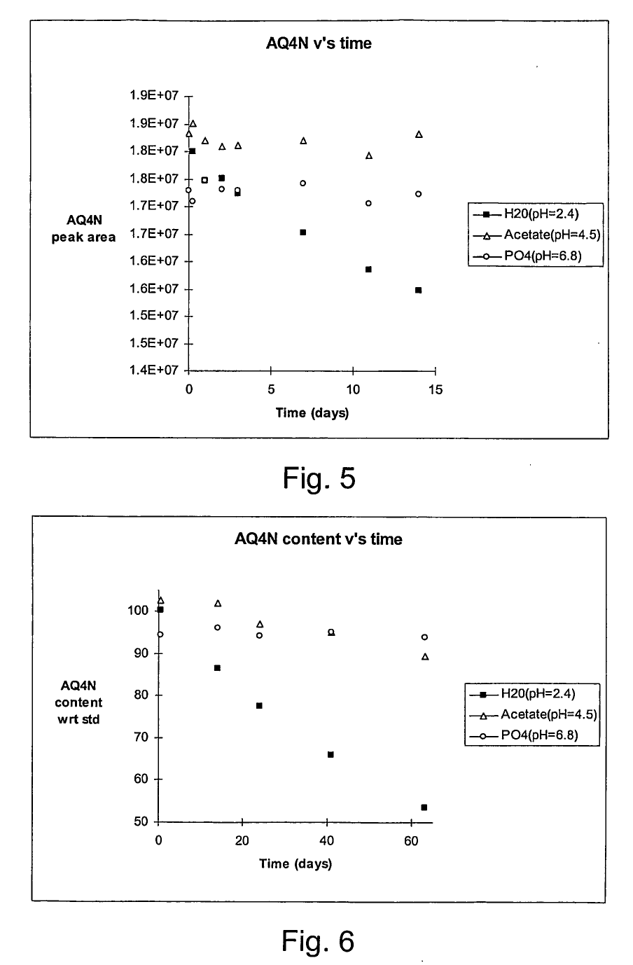

FIG. 5 shows the decrease in AQ4N over incubation time in 5 mg/ml solutions incubated at 40° C. for 14 days;

FIG. 6 shows the decrease in AQ4N over incubation time in 5 mg/ml solutions incubated at 40° C. for 63 days;

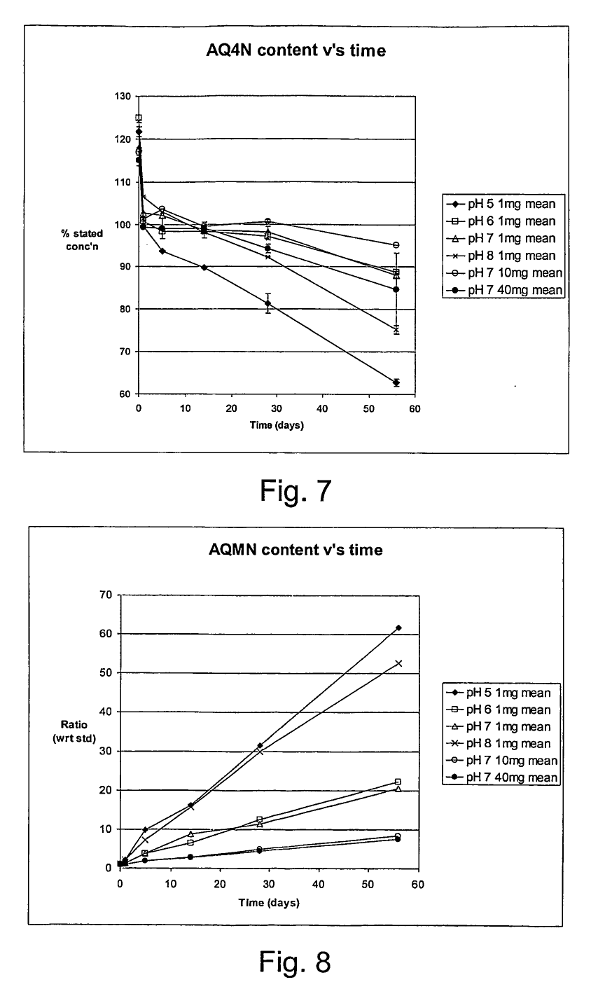

FIG. 7 shows the decrease in AQ4N over incubation time at different pH values and concentrations;

FIG. 8 shows the increase in AQMN over incubation time at different pH values and concentrations;

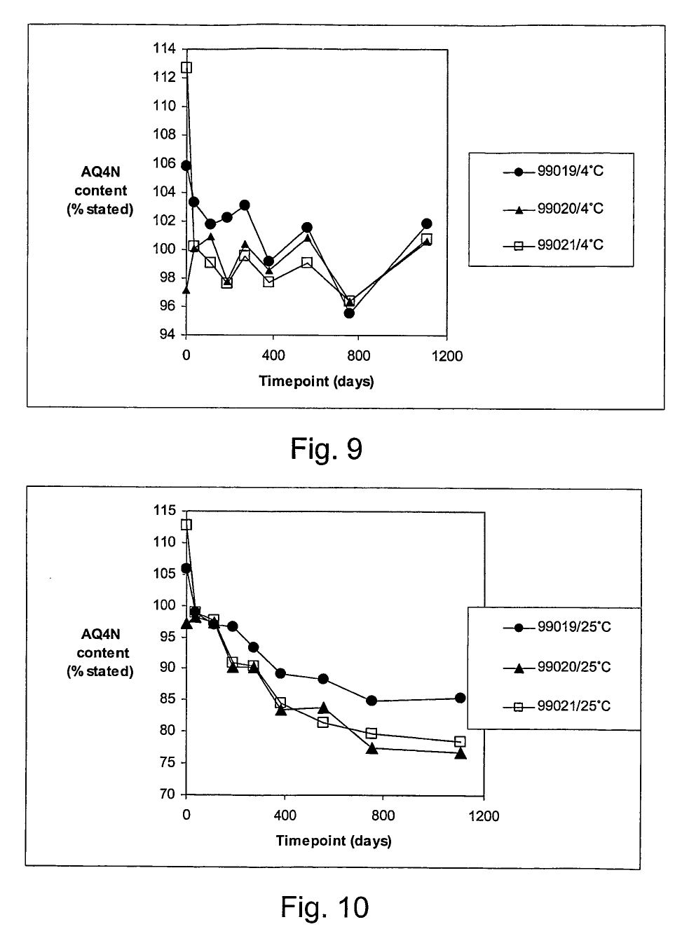

FIG. 9 shows the AQ4N content of freeze dried formulations against time when stored at 4° C.;

FIG. 10 shows the AQ4N content of freeze dried formulations against time when stored at 25° C.;

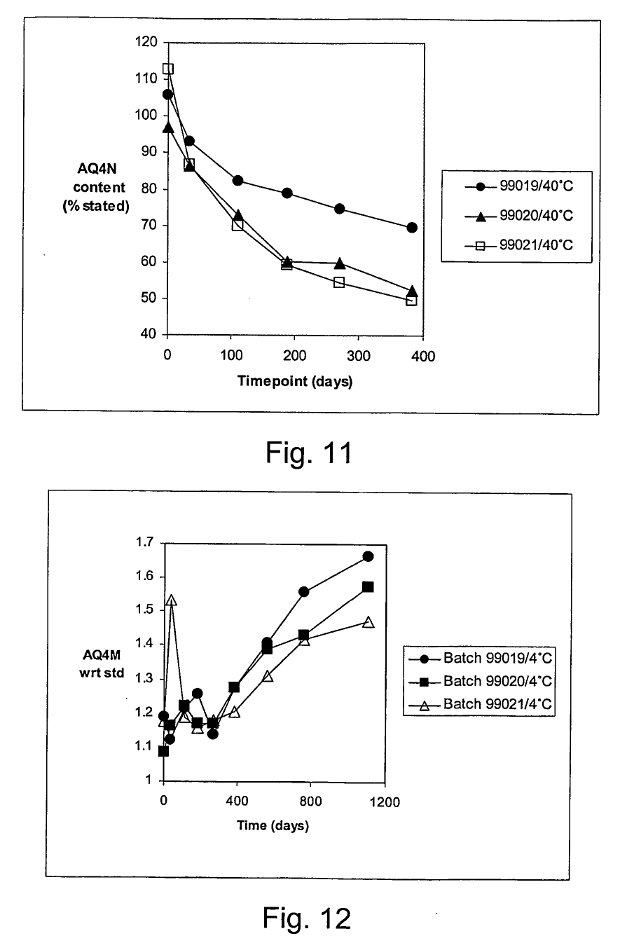

FIG. 11 shows the AQ4N content of freeze dried formulations against time when stored at 40° C.;

FIG. 12 shows changes in the AQ4M content in different freeze dried batches at 4° C.;

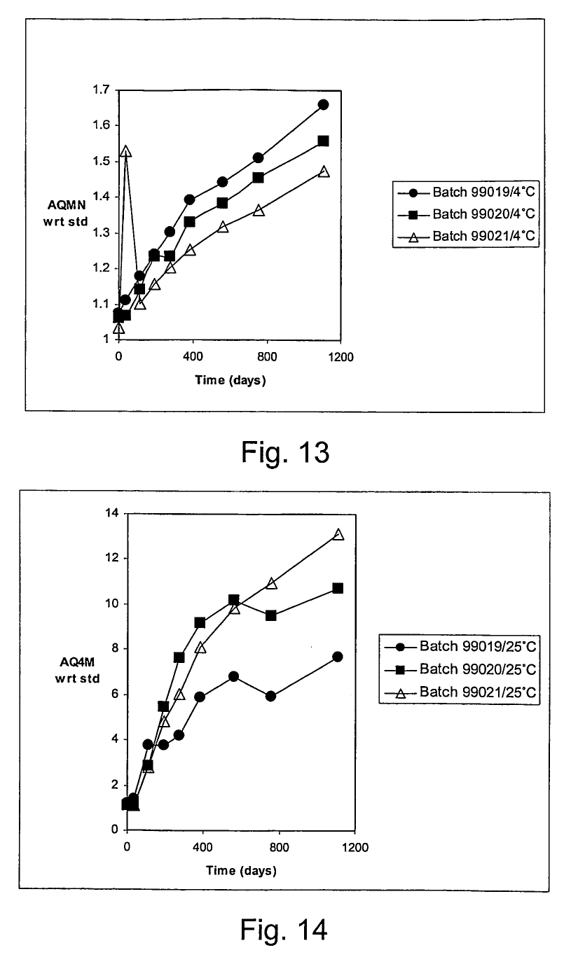

FIG. 13 shows changes in the AQMN content in different freeze dried batches at 4° C.;

FIG. 14 shows changes in the AQ4M content in different freeze dried batches at 25° C.;

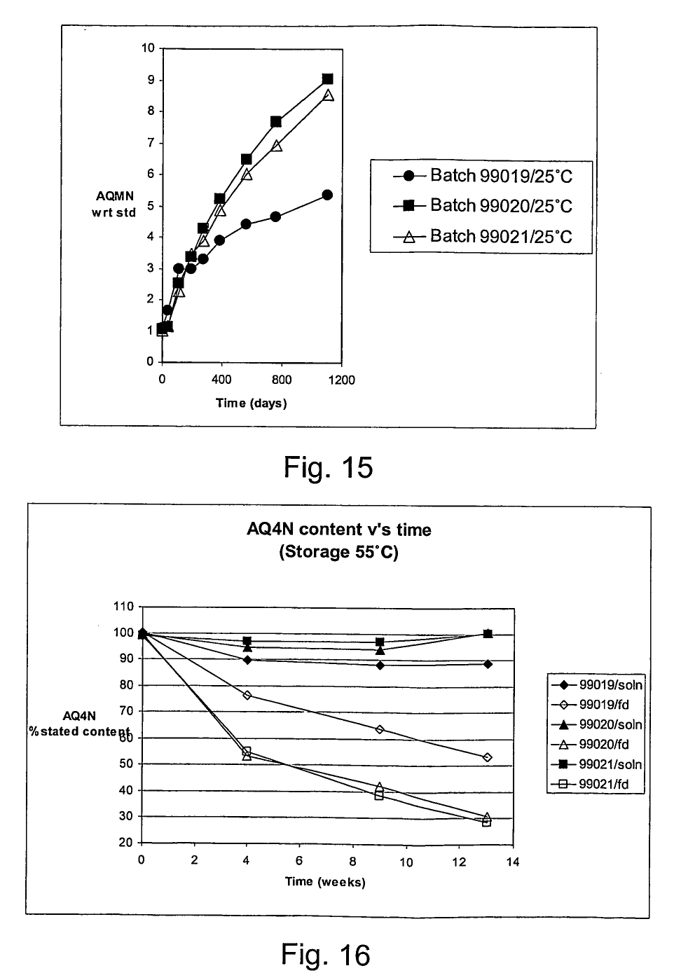

FIG. 15 shows changes in the AQMN content in different freeze dried batches at 25° C.;

FIG. 16 decrease in AQ4N content of freeze dried and solution formulations against time when stored at 55° C.;

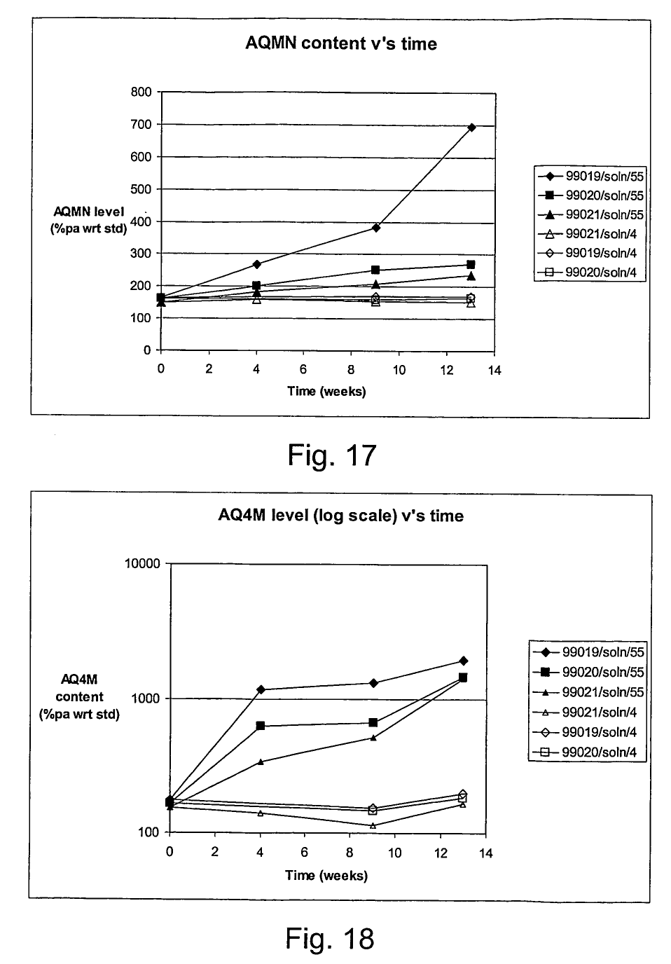

FIG. 17 shows the increase in AQMN content of solution formulations against time when stored at 4 and 55° C.;

FIG. 18 shows the increase in AQ4M content of solution formulations against time when stored at 4 and 55° C.;

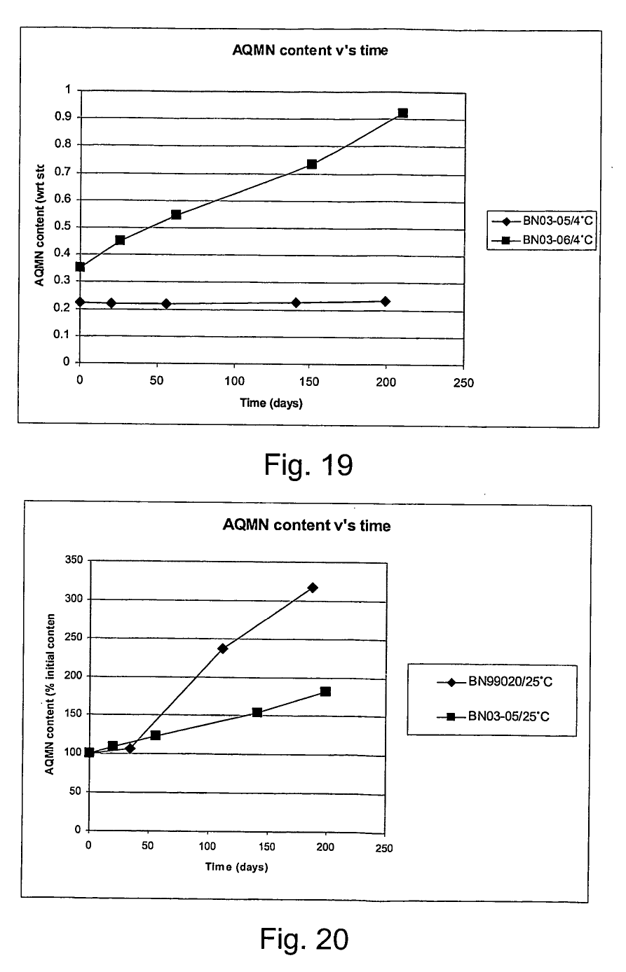

FIG. 19 shows the increase in AQMN content of solution formulations against time when stored at 4° C.;

FIG. 20 shows the increase in AQMN content of solution formulations against time when stored at 25° C.;

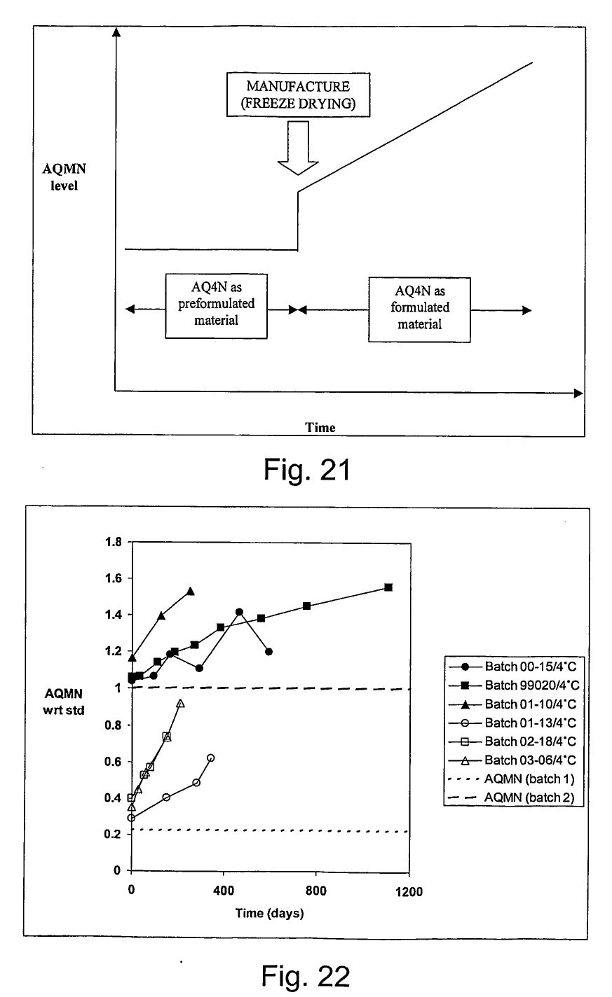

FIG. 21 shows how AQMN increase is observed in two ways;

FIG. 22 shows the increase in AQMN content of freeze dried formulations against time in several batches when stored at 4° C.;

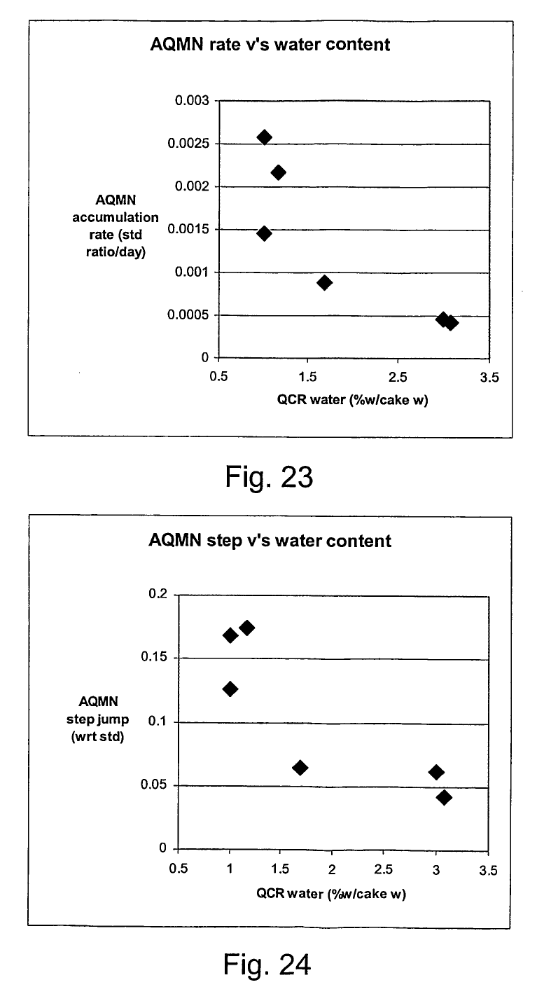

FIG. 23 shows the degradation rate (as measured by the slope of AQMN increase when fitted to a linear plot) against the water content of the batches at 4° C.; and

FIG. 24 shows the step increase in AQMN level during manufacture against the water content of the batches at 4° C.

### EXAMPLES

### Example 1

### Demonstration of the Effect of pH on the Physico-Chemical Properties of AQ4N

Changes in the pH of a solution of AQ4N dihydrochloride were monitored to demonstrate the degradation of AQ4N into AQMN. The pH curves are shown in FIGS. 1 and 2. FIG. 1 shows a clear dissociation at between pH 7.7 and pH 9.4, and this equates to the dissociation events shown in FIG. 2 at approximately 2 molar equivalence. A low pH dissociation event can be observed, speculatively assigned to a pH between 4.1 and 4.6 where the molar equivalence is between 0.95 and 1.15.

### Example 2

### Demonstration of the Cytotoxicity of AQMN

The toxicity of a pure sample (99.3%) in the P388 system of AQ4N and AQMN were determined and the results obtained are presented in Table 2.

TABLE 2
AQ4N and AQMN cytotoxicity values

| | | Relative toxicity |
|---|---|---|
| Compound | IC₅₀ P388 (nM) | (normalised to AQ4N) |
| AQ4N | 410 | 1.0 |
| AQMN | 77 | 5.2 |

Based on these data, AQMN has a cytotoxicity which is at least 5 times greater than that of AQ4N in the same system. The “greater than” modifier is required since all samples of AQ4N contain substantial percentages of AQMN, which will affect the toxicity result.

### Example 3

### Demonstration of the Instability of AQ4N Dihydrochloride in Solution-Accumulation of AQMN

The degradation of AQ4N was investigated using 5 mg/ml solutions of AQ4N at a pH of 2.4, 4.5 and 6.8, which equated to water, 20 mM sodium acetate buffer and 20 mM sodium orthophosphate buffer, respectively. The primary degradation pathway of AQ4N is its conversion to AQMN. The increase in AQMN concentration in 5 mg/ml solutions incubated at 40° C. over an intermediate time period (14 days) is shown in FIG. 3.

The degradation rates equate to a 0.84% (w.r.t. AQ4N), 0.19% (w.r.t. AQ4N) and 0.02% (w.r.t. AQ4N) increase in AQMN content per day under these conditions.

Using linear regression and cross correlating with the known quantity of AQMN in the material used as a standard these data indicate the accumulation rates of AQMN presented in Table 3.

TABLE 3
Accumulation of AQMN

| | H₂O | Acetate | PO₄ |
|---|---|---|---|
| AQMN increase per day | 0.84% | 0.19% | 0.02% |

A similar trend was observed in the data after 63 days, as shown in FIG. 4. The rate of accumulation (calculated in the same way as above) shows that in the phosphate buffer AQMN increases by 0.6% per month.

### Example 4

### Demonstration of the Instability of AQ4N Dihydrochloride in Solution-Degradation of AQ4N

The effect of pH on the stability of AQ4N was determined by investigating AQ4N degradation in different solutions. The solutions chosen were again distilled water, 20 mM sodium acetate buffer (pH=4) and 20 mM sodium phosphate buffer (pH=7). After preparation of the 5 mg/ml AQ4N buffered solutions the pH was corrected to the required pH of the buffer. The final pH values were 2.4, 4.5 and 6.8 for the distilled water, 20 mM sodium acetate buffer and 20 mM sodium phosphate buffer, respectively. The samples were incubated at 40° C. and sampled at regular intervals. Assay was carried out by sample dilution followed by HPLC analysis.

After 14 days an interim analysis was carried out with the data being shown in FIG. 5.

The solutions were further incubated at 40° C. for a total of 63 days. The final graph is shown in FIG. 6. The values obtained for the AQ4N contents are consistent with the quantities of AQ4N weighed into the original samples (within experimental error).

### Example 5

### Demonstration of the Instability of AQ4N as a 40 mg/ml Solution in 10 mM sodium Phosphate Buffer at pH=7.4 to ‘Standard’ Autoclave Conditions

A solution of AQ4N was prepared at 40 mg/ml in 10 mM sodium phosphate buffer at pH=7.4. Samples of this solution were analysed before and after a standard autoclave cycle (121° C. for 15 minutes). The post autoclave samples showed a decrease in AQ4N concentration of 7.3%. Corresponding increases were seen with associated impurities.

### Example 6

### Demonstration of the Relationship Between the Stability of AQ4N and pH at Various Concentrations

Several solutions of AQ4N were prepared as in sodium phosphate buffer (10 mM) with different concentrations of AQ4N at different pH values and the results obtained are presented in Table 4.

TABLE 4
pH of AQ4N solutions

| | | |
|---|---|---|
| | AQ4N conc. | Solution pH |
| Solution | (mg/ml) | at preparation |

| | | | |
|---|---|---|---|
| pH 5 | 1 mg | 1.0 | 5.14 |
| pH 6 | 1 mg | 1.0 | 6.18 |
| pH 7 | 1 mg | 1.0 | 7.03 |
| pH 8 | 1 mg | 1.0 | 8.43 |
| pH 7 | 10 mg | 10.0 | 7.48 |
| pH 7 | 40 mg | 39.8 | 7.48 |

The samples were assayed before incubation and then placed in a 55° C. incubator. The solutions were re-sampled after one day, one week, two weeks, four weeks and eight weeks.

The results in terms of the AQ4N content are shown in FIG. 7 and the results in terms of the increase in the AQMN in FIG. 8.

These results show that between 10 and 40 mg/ml (at pH 7.4) there is little effect of concentration change on the degradation rate. These results also show that the stability pH profile of AQ4N runs as follows:
7.4 (stable)<(7.0≅6.2)<(8.4≅5.1) (unstable)

### Example 7

### Demonstration of the in Stability of AQ4N as a Freeze Dried Formulation at Various pH Values

Three lyophilised batches of AQ4N (40 mg/ml, sodium phosphate buffer 10 mM at pH=6.0, 7.0 and 8.0) were manufactured as BN99019, BN99020 and BN99021, respectively. Samples for these batches were stored at 4° C., 25° C. and 40° C. and analysed at various time points according to Table 5.

TABLE 5
Time points of stability batches (BN99019, BN99020
and BN99021) under different storage temperatures

| | | Batch | | | | | | | | |
|---|---|---|---|---|---|---|---|---|---|---|
| | | 99019 | | | 99020 | | | 99021 | | |
| Time point | Time point | Temp. | | | | | | | | |
| (months) | (days) | 4° C. | 25° C. | 40° C. | 4° C. | 25° C. | 40° C. | 4° C. | 25° C. | 40° C. |
| 0 | 0 | ✓ | ✓ | ✓ | ✓ | ✓ | ✓ | ✓ | ✓ | ✓ |
| 1 | 34 | ✓ | ✓ | ✓ | ✓ | ✓ | ✓ | ✓ | ✓ | ✓ |
| 3 | 111 | ✓ | ✓ | ✓ | ✓ | ✓ | ✓ | ✓ | ✓ | ✓ |
| 6 | 188 | ✓ | ✓ | ✓ | ✓ | ✓ | ✓ | ✓ | ✓ | ✓ |
| 9 | 271 | ✓ | ✓ | ✓ | ✓ | ✓ | ✓ | ✓ | ✓ | ✓ |
| 12 | 383 | ✓ | ✓ | ✓ | ✓ | ✓ | ✓ | ✓ | ✓ | ✓ |
| 18 | 559 | ✓ | ✓ | N/A | ✓ | ✓ | N/A | ✓ | ✓ | N/A |
| 24 | 756 | ✓ | ✓ | N/A | ✓ | ✓ | N/A | ✓ | ✓ | N/A |
| 36 | 1106 | ✓ | ✓ | N/A | ✓ | ✓ | N/A | ✓ | ✓ | N/A |

At each time point AQ4N content, AQ4N related impurity levels, water content, pH and osmolality were investigated. The latter three are not further discussed.

The AQ4N content levels for the pH=6.0, 7.0 and 8.0 formulations as BN99019, BN99020 and BN99021, respectively, are shown against time in FIGS. 9 to 11 for individual temperatures. FIG. 9 shows the AQ4N content when stored at 4° C., FIG. 10 at 25° C. and FIG. 11 at 40° C.

The degradation of the AQ4N (as measured by the decrease of the parent compound) can clearly be seen at 25 and 40° C. BN99019 (pH=6.0) appears to be the most stable.

The related impurity levels were plotted for three batches under the 4 and 25° C. storage conditions.

The degradation profile of the AQ4N manufactured product indicate that the main degradants are AQ4M and AQMN. The related impurity levels for the pH=6.0, 7.0 and 8.0 formulations as BN99019, BN99020 and BN99021, respectively, are shown in FIGS. 12 to 15. FIGS. 12 and 13 respectively show the AQ4M and AQMN changes at 4° C., and FIGS. 14 and 15 show the AQ4M and AQMN changes at 25° C.

The 25° C. results confirm that the pH=6.0 formulation is the most stable. However, it appears as if the degradant levels in the BN99019 samples stored at 4° C. are slightly higher than the pH=7.0 and 8.0 formulations.

### Example 8

### Demonstration of the Relationship Between the Stability of AQ4N and pH at 40 mg/ml

Previous work on the stability of AQ4N at low concentrations (5 mg/ml) (see Examples 3 and 4 above) suggested that at neutral pH the stability of the AQ4N molecule is high. This experiment was designed to investigate how stable the formulated AQ4N product is as a reconstituted formulation (at 40 mg/ml). Formulated AQ4N material was prepared at 40 mg/ml in 10 mM sodium phosphate buffer at pH 6.0, 7.0 and 8.0 and freeze dried. These batches were placed on stability for 3 years as an indication of the stability of the freeze dried material (see Example 7). After that time the vials stored at 4° C. (where the loss of AQ4N was show to be minimal) were reconstituted in 2 ml of distilled water. (The pH of the last time point on stability was 5.916, 6.946 and 7.928, respectively.) Two vials were assayed for AQ4N, AQMN and other related impurities after reconstitution. Both freeze dried and reconstituted vials were stored as specified in Tables 6 to 8.

TABLE 6
Formulated solutions at pH 6.0

| | 4° C. | 55° C. | 55° C. |
|---|---|---|---|
| Time (weeks) | (solution) | (solution) | (f/d vial) |
| 4 | — | 2 vials | 2 vials |
| 9 | 1 vials | 2 vials | 2 vials |
| 13 | 2 vials | 2 vials | 2 vials |

TABLE 7
Formulated solutions at pH 7.0

| | 4° C. | 55° C. | 55° C. |
|---|---|---|---|
| Time (weeks) | (solution) | (solution) | (f/d vial) |
| 4 | — | 2 vials | 2 vials |
| 9 | 2 vials | 2 vials | 2 vials |
| 13 | 2 vials | 2 vials | 2 vials |

TABLE 8
Formulated solutions at pH 8.0

| | 4° C. | 55° C. | 55° C. |
|---|---|---|---|
| Time (weeks) | (solution) | (solution) | (f/d vial) |
| 4 | 2 vials | 2 vials | 2 vials |
| 9 | 2 vials | 2 vials | 2 vials |
| 13 | 2 vials | 2 vials | 2 vials |

The results for AQ4N content for the samples stored at 55° C. are shown in FIG. 16.

These data confirm that at pH 6.0 the freeze dried material is more stable and the equivalent pH 7 or 8 material, but also shows that formulated AQ4N is more stable as a solution than as a freeze dried preparation. The pH stability profile for solutions of AQ4N in this experiment is shown to be:
(6.9≅7.9) (stable)<5.9 (unstable)

The results for the degradants (AQMN and AQ4M) are shown in FIGS. 17 and 18 for the solutions samples.

A direct numerical comparison of the degradation rates of the Examples 9 and 6 is not possible since different AQ4N raw materials were used as standards and samples for these individual experiments.

### Example 10

### Demonstration of Effect of Freeze Drying (Against Solution Preparation) on the Quality of AQ4N Product

Formulated AQ4N material was prepared at 40 mg/ml in 10 mM sodium phosphate buffer at pH 7.0. A portion of this solution was filled into ampoules and the remained into vials and freeze dried according to the cycle as specified in Tables 9 and 10.

TABLE 9
Temperature Profile

| | | | | | Cumulative |
|---|---|---|---|---|---|
| | Temperature | Time | | | Time |
| Stage | (° C.) | Hours | Minutes | Seconds | HH.MM.SS* |
| 00 | 20 | 3 | 00 | 00 | 00.00.00 |
| 01 | −40 | 2 | 00 | 00 | 03.00.00 |
| 02 | −40 | 5 | 00 | 00 | 05.00.00 |
| 03 | −5 | 15 | 00 | 00 | 10.00.00 |
| 04 | −10 | 5 | 00 | 00 | 25.00.00 |
| 05 | 10 | 5 | 00 | 00 | 30.00.00 |
| 06 | 10 | 0 | 00 | 00 | 35.00.00 |
| | Total Time = | 35 | 00 | 00 | |

*Cumulative time at start of programme section

TABLE 10
Cycle positions

| | Time | | |
|---|---|---|---|
| Cycle Position | Hours | Minutes | Seconds |
| Initiation | 00 | 00 | 01 |
| Freezing | 4 | 00 | 00 |
| Evacuation | 0 | 29 | 58 |
| Primary Drying | 23 | 30 | 00 |
| Secondary Drying | 7 | 00 | 00 |
| Cycle End | 0 | 00 | 01 |
| Total Time = | 35 | 00 | 00 |

The AQMN content of the samples before and after freeze drying (ampoule fill and vial fill, respectively) were measured as shown in Table 11.

TABLE 11
AQMN content

| | AQMN content |
|---|---|
| Sample | (% peak area w.r.t. std.) |
| Ampoule | 22.2% |
| Vial | 35.2% |

The AQMN content of the relevant AQ4N raw material compared to the standard is 22.6%. This indicates that the AQMN is generated during the freeze drying cycle, and the elimination of the lyophilisation step improves the overall quality of the product.

### Example 11

### Demonstration of the Effect of Temperature on the Stability of AQ4N

The AQ4N solution (liquid-filled) ampoules prepared in Example 10 were tested for stability by incubation at 4° C. and 25° C. At various time points the AQ4N, AQMN and related impurity contents of the vials were assayed by HPLC. The AQMN contents for the ampoules (designated BN03-05) stored at 4° C. are shown in FIG. 19 as compared to the AQ4N freeze dried vials (BN03-06), also prepared in Example 10 (and stored at 4° C.). The AQMN contents for the ampoules stored at 25° C. are shown in FIG. 20 as compared to the equivalent AQ4N freeze dried vials (BN99020, pH=7), prepared in Example 7, and stored at 25° C. (The data for both groups are only shown for comparable times at approximately 200 days.)

It should be noted that since BNO3-05 and BN99020 were prepared from different raw material batches (and so contained different levels of AQMN initially) the y-axis is expressed as a percentage of the initial AQMN content of the final product batch).

### Example 12

### Demonstration of the Stability of AQ4N at at 4° C.

A further illustration of the stability of the aqueous formulation was demonstrated by an additional batch of AQ4N solution (liquid-filled) ampoules (BN03-12) prepared in a similar way to those in Example 10. However, BN03-12 was prepared at with a larger volume 200 mg in 5 ml of solution. The batch was placed at 4° C. and analysed at various timepoints to allow suitable expiry date extension. The AQMN levels of this material were consistent with Example 11, where the AQMN content value (w.r.t. the standard) increased from 0.226 after manufacture to 0.232 after 162 days at 4° C.

### Example 13

### Demonstration of the Instability of AQ4N as a Freeze Dried Formulation with Respect to Water Content

Several batches of the AQ4N freeze dried product (40 mg/ml, 10 mM sodium phosphate buffer at pH=7.0) were prepared in a similar or identical manner to that described above.

AQMN increase is observed in two ways: a step increase in AQMN content during manufacture (see Example 10 above) and gradual increase over time (see FIG. 21).

The AQMN increase at 4° C. in several batches is shown in FIG. 22.

The straight lines on the graph represent the AQMN level found in the raw material which was used to manufacture particular batches. (Raw material 1 was used for batches 01-13, 02-18 and 03-06. Raw material 2 was used for batches 99020, 00-15 and 01-10.)

The water content of the batches (as measured by Karl Fisher titration at QC/post manufacture testing) was plotted against the degradation rate at 4° C. (as measured by the slope of AQMN increase when fitted to a linear plot) and is given in FIG. 23.

The water content of the batches (as measured by Karl Fisher titration at QC/post manufacture testing) was plotted against the step increase in AQMN level during manufacture (specifically freeze drying) and is given in FIG. 24.

Both these graphs show the correlation between the degradation of the product (during and after manufacture) and the amount of water in the freeze dried cake after manufacture. This appears to be a function of freeze drying process itself which is indicated by the water content.

### Example 14

### Preparation of Stable, Sterile Aqueous Solutions

Preparation and Qualification of Equipment and Environment

Equipment and containers for the production of the AQ4N final product were treated appropriately (including dry heat sterilisation, autoclave sterilisation and IPA disinfection) for aseptic manufacture of a sterile product. The clean rooms and laminar air flow cabinets used during the preparation and production were monitored (using settle and contact plates, air pressure differential and particle counts) in a manner appropriate for the manufacture of a sterile product. Balances used for weighing during the production process were calibrated with traceable weights immediately prior to manufacture.

Buffer Preparation

Using a three-place balance, 3.450 g of sodium dihydrogen orthophosphate 1-hydrate (monobasic) was weighed into a 600 ml beaker and 8.900 g of di-sodium hydrogen orthophosphate 2-hydrate (dibasic) was weighed into a 1000 ml beaker. 400 mL of water for irrigation (WFI) was added to the monobasic and 800 mL to the dibasic beakers respectively. A magnetic follower was added and allowed to stir for 15 minutes. Each solution was aseptically transferred to an individual 500 mL (monobasic) and 1000 mL (dibasic) volumetric flask. Each flask was appropriately labelled, made to volume and stoppered.

390 ml of Monobasic solution and 610 mL of Dibasic solution were added to a 2000 mL beaker using 500 mL and 1000 mL measuring cylinders. These were mixed and labelled as 0.05 M phosphate buffer pH 7.0.

375 mL of the pH 7.0 buffer was transferred into a 500 mL volumetric flask by weight. (weight required is 376.58 g since the density of the solution has been determined to be 1.0042 g/ml) and used for solubilisation of AQ4N.

Processing of the Bulk Solution

30.00 g of AQ4N (as a dihydrochloride salt) was added into a 1000 mL beaker. The weight was recorded and a magnetic stirrer bar added.

43.5 mL of 2M sodium hydroxide was added via a 50 mL syringe and a 5 mL syringe into a 500 mL beaker and make to an approximately 150 mL volume with WFI. The 150 mL of the NaOH was added to the AQ4N powder. Approximately 225 mL of pH 7.0 buffer was added to the AQ4N mixture ensuring that all the AQ4N in the 1000 mL beaker was solubilised with buffer during this procedure. This solution was then transferred to the 2000 mL beaker, rinsed with the remaining buffer solution and allowed to stir for 15 minutes. The AQ4N solution was made up to approximately 600 ml volume with WFI. The pH was established and adjusted to 7.0±0.1 if required. WFI was added to the AQ4N solution until a target weight of 760 grams was obtained.

Filtration of Bulk Solution

A peristaltic pump with clean, sterile, inert tubing was aseptically assembled to pump the AQ4N solution from the bulk solution, through two Millipak™ 20 filters and into a clean, treated, sterile Schott bottle assembly (including filling lines and vent filters).

Ampoule Filling and Stoppering

If ampoule filling was required, ampoules were purchased, prepared, treated and opened to a suitable quality standard for pharmaceutical use prior to AQ4N solution preparation. The filtered AQ4N solution was dispensed into ampoules using a peristaltic pump, with clean, sterile, inert tubing. Dispensing was carried out by volume, but checked by weight before filling and at regular intervals during manufacture (nominally after every fiftieth ampoule, but this would vary according to batch size.). The density of the solution was previously established. The ampoules were sealed manually by gas-air flame in the clean room.

1. A stable, sterile aqueous solution of a compound of formula (I): 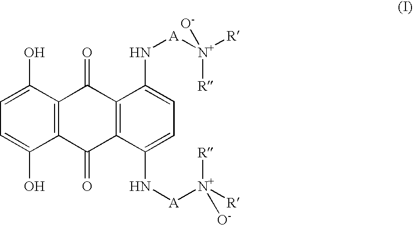
in which A is a C alkylene group with a chain length between NH and N(O)R′R″ of at least 2 carbon atoms and R′ and R″ are each separately selected from C₁₋₄ alkyl groups and C₂₋₄ hydroxyalkyl and C₂₋₄ dihydroxyalkyl groups in which the carbon atom attached to the nitrogen atom does not carry a hydroxy group and no carbon atom is substituted by two hydroxy groups, or R′ and R″ together are a C₂₋₆ alkylene group which with the nitrogen atom to which R′ and R″ are attached forms a heterocyclic group having 3 to 7 atoms in the ring,
in a unit dosage form in a sealed container, said solution having a concentration of the compound of formula (I) up to 150 mg/ml and a pH in the range of 5 to 9.

2. A solution as claimed in claim 1 in which the pH of the solution is in the range of 5.0 and 8.4.

3. A solution as claimed in claim 2 in which the pH of the solution is in the range of 6.0 to 8.0.

4. A solution as claimed in claim 1 in which the compound of formula (I) is present at a concentration of between 0.1 and 100 mg/ml.

5. A solution as claimed in claim 1 in which A is a straight chain alkylene group.

6. A solution as claimed in claim 1 in which A is ethylene.

7. A solution as claimed in claim 1 in which R′ and R″ are straight chain alkyl groups or hydroxy-substituted alkyl groups.

8. A solution as claimed in claim 7 in which R′ and R″ are each CH₃ or CH₂CH₃.

9. A solution as claimed in claim 8 in which each group of formula NH-A-N(O)R′R″ is group of formula NH—(CH₂)₂—N(O)(CH₃)₂.

10. A solution as claimed in claim 1 formulated in a mixture containing additional components so that the pH of the solution is buffered to be in the range of 5 to 9.

11. A solution as defined in claim 1 for use in therapy.

12. A process for the preparation of a solution as defined in claim 1 comprising introducing a stable, sterile aqueous solution of the compound of formula (I) into a container and sealing the container, in which the solution is prepared without a freeze drying step.
